(19) United States
(12) Patent Application Publication
(10) Pub. No.: US 2008/0249419 A1
(43) Pub. Date: Oct. 9, 2008

(54) TIME-OF-FLIGHT TRIANGULATION BASED METHODS OF DEVICE SPATIAL REGISTRATION FOR MULTIPLE-TRANSDUCER THERAPEUTIC ULTRASOUND SYSTEMS

(76) Inventors: K. Michael Sekins, Yarrow Pt., WA (US); Charles Emery, Issaquah, WA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/955,310

(22) Filed: Dec. 12, 2007

Related U.S. Application Data
(60) Provisional application No. 60/869,702, filed on Dec. 12, 2006.

(51) Int. Cl. A61B 8/00

(52) U.S. Cl. 600/463

(57) ABSTRACT

Embodiments disclosed herein relate to a therapeutic ultrasound device spatial registration method and apparatus. The method enables locating a tissue target and surrounding tissue structures affecting the available acoustic beam paths to the target, relative to each individual therapeutic and diagnostic (e.g., imaging) transducer, in a device comprising a plurality of such transducers. By locating the target and tissue structures relative to each therapeutic transducer and/or array (e.g., in “local” coordinates) assists in transforming the locations relative to any transducers, enabling determination of which therapeutic transducers to use to treat the target, and specification of their respective ultrasound powers or energy, focal locations and beam patterns. The registration method employs a plurality of emitter and receiver acoustic sensors on, respectively, a plurality of imaging and therapeutic transducer device panels.

### RELATED APPLICATIONS

This application claims priority to, and incorporates by reference in its entirety, U.S. Patent Application No. 60/869,702, filed Dec. 12, 2006, entitled “TIME-OF-FLIGHT TRIANGULATION BASED METHODS OF DEVICE SPATIAL REGISTRATION FOR MULTIPLE-TRANSDUCER THERAPEUTIC ULTRASOUND SYSTEMS”.

### STATEMENT REGARDING FEDERALLY SPONSORED R&D

The U.S. Government has a paid-up license in this invention and the right in limited circumstances to require the patent owner to license others on reasonable terms as provided for by the terms of Contract No. W81XWH-06-C-0061 entitled “Noninvasive Acoustic Coagulation System for Life Threatening Battlefield Extremity Wounds” awarded by the Defense Advance Research Projects Agency (DARPA).

### BACKGROUND OF THE INVENTION

1. Field of the Invention

The present invention relates to ultrasound arrays for therapeutic and diagnostic use.

2. Description of the Related Art

Certain injurious events result in bleeding penetration wounds in the limbs of the human body, for example military combat bullet and shrapnel wounds, vehicular accidents, and insertion of penetrating devices (needles and catheters) into tissue during medical procedures, such as blood vessels and/or organs. Following such injuries, it is desirable to rapidly stop the bleeding from these wounds (hemostasis), especially bleeding from puncture wounds of significant blood vessels, and to do so in an efficient manner, minimizing time and effort. One method for causing hemostasis is through the use of therapeutic ultrasound. Therapeutic ultrasound can also be used to treat tumors or other unwanted growths within the body. There is a need for improved ultrasound systems that can be used in therapeutic and diagnostic applications such as hemostasis.

### SUMMARY OF THE INVENTION

In some embodiments, an ultrasound device is provided, including a first rigid component comprising an array of ultrasound transducers, the first rigid component comprising at least one ultrasound transducer configured to transmit an ultrasound pulse; a second rigid component comprising an array of ultrasound transducers, the second rigid component comprising at least one ultrasound transducer configured to receive the transmitted pulse; and a processor configured to determine the distance between the ultrasound transducer configured to transmit the ultrasound pulse and the ultrasound transducer configured to receive the transmitted pulse.

In some embodiments, a method of transmitting focused ultrasonic energy is provided, including determining a first location of a target relative to a first device component using ultrasound imaging; emitting a signal from an ultrasound transducer located on said first device component; detecting said signal at a second device component; determining a second location of the target relative to the second device component based at least in part on the detected signal; and applying high intensity focused ultrasonic energy from a therapeutic transducer array located on the second device component to the target.

In some embodiments, a method of transmitting focused ultrasonic energy is provided including determining a first location of a target relative to a first device component using ultrasound imaging; emitting a signal from an ultrasound transducer located on a second device component; detecting said signal at said first device component; determining a second location of the target relative to the second device component based at least in part on the detected signal; and applying high intensity focused ultrasonic energy from a therapeutic ultrasound transducer array located on the second device component to the target.

In some embodiments, a method of transmitting focused ultrasonic energy is provided, including positioning an ultrasound device comprising a therapeutic ultrasound array proximate to a human body; emitting a signal from a first component of said ultrasound device into the body; detecting said signal at a second component of said ultrasound device; determining a speed of sound or signal attenuation in the body based at least in part on the detected signal; and applying high intensity focused ultrasonic energy from said therapeutic transducer array into the body, wherein at least one characteristic of the energy is adjusted based on the determined speed of sound or signal attenuation.

In some embodiments, a method of manufacturing an ultrasound device is provided, including positioning an emitter configured to transmit an ultrasound signal on a first rigid device component; and positioning a receiver configured to receive the transmitted ultrasound signal on a second rigid device component that is coupled directly or indirectly to the first rigid device component, wherein the second rigid device component is movable with respect to the first rigid device component.

### BRIEF DESCRIPTION OF THE DRAWINGS

FIG. 1 is a schematic of adjacent therapeutic transducer panels relative to a target.

FIG. 2 is a schematic of a device comprising therapeutic and imaging transducer panels.

FIG. 3 is a cross-sectional schematic of a cuff-shaped device depicted on a human limb, whereby the device comprises therapeutic and imaging transducer panels.

FIG. 4 is a schematic of three transducer panels in a deep bleeder acoustic coagulation cuff having panel acoustic localization sensors.

FIG. 5 is a schematic of the panels of FIG. 4 illustrating spatial registration using sensors on a transducer panel.

FIG. 6 is a schematic of the panels of FIG. 4 illustrating spatial registration using sensors on an imaging panel.

### DETAILED DESCRIPTION OF THE PREFERRED EMBODIMENT

Certain medical therapeutic ultrasound systems may be configured to exploit the advantages of using, either sequentially or simultaneously, multiple therapeutic transducers to deliver acoustic energy to targets (e.g., tissue targets or vessels). Furthermore, some therapeutic ultrasound systems may be advantageously guided (i.e., for targeting tissues to be treated) by ultrasound images (or other lower energy acoustic signal information). In addition, such ultrasound guided systems may benefit from using the ultrasound image of signal information from multiple “diagnostic” ultrasound transducers. Embodiments disclosed herein relate to identifying an absolute or relative position and spatial orientation of a device component (e.g., a panel comprising one or more therapeutic or diagnostic transducers or transducer arrays) using one or more point-like acoustic sensors (e.g., an emitter and/or receiver), so that such position and orientation information can be used for setting a variety of system parameters, such as determining which therapeutic transducers will, or should be used to, transmit therapeutic energy toward the tissue target and/or determining the power and the spatial profile (e.g., focal position) of the therapeutic beams.

### Therapeutic Ultrasound Devices

In some embodiments, therapeutic ultrasound systems are provided that comprise multiple transducers, both therapeutic and diagnostic. The transducers may be mechanically connected or mechanically positioned in desired spatial relationships to each other. For example, an ultrasound system may comprise a plurality of panels, each panel comprising an array of transducers. The panels may then be mechanically coupled to each other to form a larger system. By virtue of this design, the transducers may be arranged in specific configurations, such as configurations where the collection of the transducers surrounds, or partially surrounds, the portion of the body to be treated. The transducers may be positioned or configured to surround a target or treatment area and/or to form an acoustic enclosure (e.g., a cuff on a limb with beams pointed into the tissue) or a conformal partial enclosure (e.g., a “blanket” or “patchwork” of transducers on, and partially encompassing, the trunk, head, neck, and so on) around a target. Therapeutic transducers and arrays of transducers may be dispersed around a portion of the body to be treated enabling increased concentration of acoustic energy delivery (by power administered to a medium, such as a tissue, from one or more of the transducer arrays simultaneously). The therapeutic transducers may be configured to provide ultrasonic energy with enough intensity to induce hemostasis, to ablate undesirable tissue or to modify tissue in a desired way. While a single array of transducers or portion of an array may be characterized by a limited available acoustic window and/or path (which may be caused, for example, by interfering structures such as a bone), a plurality of transducer arrays may exploit more available acoustic windows and paths.

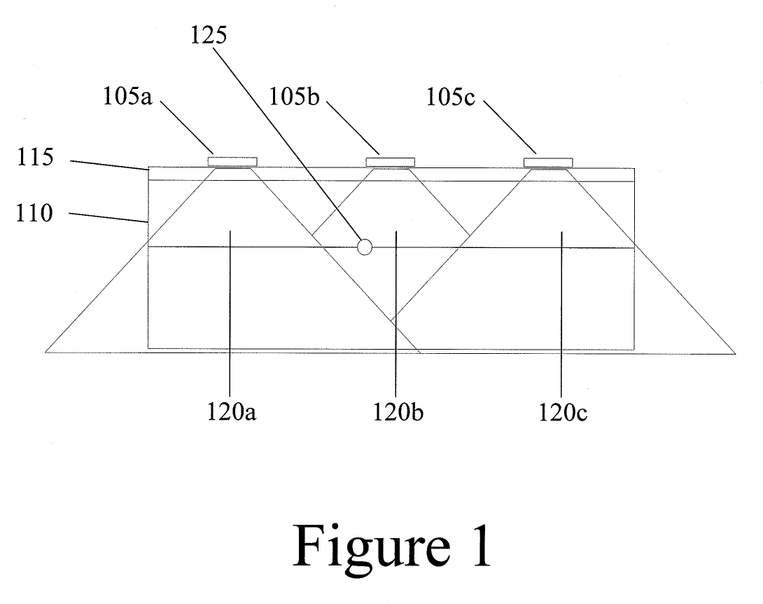

FIG. 1 shows a device incorporating a plurality of therapeutic transducers or transducer arrays 105a-c. The device may be separated from a media, such as a tissue 110 by a coupling layer 115. The coupling layer 115 may comprise, for example, a water pillow or other acoustic coupling material. Each transducer or array is capable of emitting ultrasound energy across a specific spatial region 120a-c. Ultrasound energy from a given array may be focused to a particular target region. It is desirable to strategically energize the transducers 105a-c, such that the energy is focused on a target 125, and such that the energy transmitted across and into the rest of the tissue 110 away from the target is minimized, and it is desirable to minimize the possibilities of burning skin or superficial tissue.

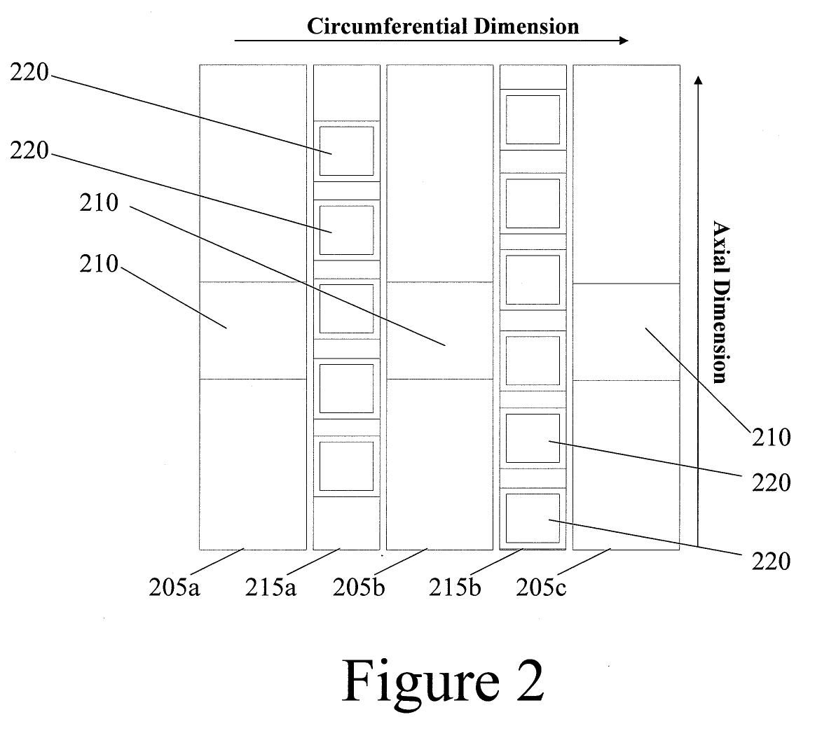

As illustrated in FIG. 2, in some embodiments, a portion of a device includes imaging array transducers (Ix) and therapeutic array transducers (Tx). Imaging transducers may be used to detect and/or localize bleeding vessels or other target tissues, and the therapeutic transducers may be used to treat the targeted area. For example, the therapeutic transducers may be used to treat, ablate or destroy tumors or fibroids, cause hemostasis and/or modify tissue (e.g., shrink collagen). The imaging transducer arrays and the therapeutic transducer arrays may be distributed among and placed on panels of the device. Alternatively, imaging and therapeutic arrays may be located on the same panel. In some embodiments, the same transducer arrays can be used for both imaging and therapy. In FIG. 2, each of the therapeutic panels 205a-c includes therapeutic transducer arrays 210, and each of the imaging panels 215a-b includes imaging transducer arrays 220. In FIG. 2, the device is shown as a relatively flat sheet. In some embodiments, the device may comprise transducers arranged in acoustic “blankets,” or “patches.” In others, the device may comprise curves. For example, the device portion of FIG. 2 may be curved such that it is comprised within a “cuff” to be formed around, for example, a body part (e.g., an arm or a leg). In this instance, the device portion of FIG. 2 may be positioned such that the panels 205a-c and 215a-b extend along the axial dimension (e.g., parallel to a body part axis, such as a limb) and the panels alternate between therapeutic panels 205a-c and imaging panels 215a-b in the circumferential dimension (e.g., going around the circumference of the body part). A cuff configuration may be used to treat bleeding regions by, for example, heating the region and/or coagulating the blood and tissue through acoustic hemostasis.

The panels 205 and 215 and the transducer arrays 210 and 220 may be of an appropriate length and width. In some embodiments, the width of the panels 205 and 215 in the circumferential dimension is substantially the same as the width of the transducer arrays 210 and 220 within the panel in the circumferential dimension. The transducer arrays 210 and 220 may be at least, for example, about 4 mm, about 6 mm, about 8 mm, about 1 cm, about 2 cm, about 3 cm, about 4 cm, about 5 cm, about 6 cm, about 7 cm, about 8 cm, about 9 cm, about 10 cm, or about 20 cm long in either the circumferential dimension or the axial dimension. In one instance, the therapeutic transducer arrays 210 are larger in one or both dimensions than the imaging transducer arrays 220. The therapeutic transducer arrays 210 may, for example, be about 8 cm and about 6 cm long in the circumferential and axial dimensions, respectively. The imaging transducer arrays 220 may be about 5 cm (e.g., 5.2 cm) long in the circumferential dimension. Panels 205 and 215 of a device may be separated by a gap. The gap may be, for example, about 0.1 mm, about 0.2 mm, about 0.5 mm, or about 1 mm.

In some embodiments, both imaging and therapeutic transducers are located on the same panel. In other embodiments, they are located on separate panels. In still other embodiments, the same transducer arrays are used for imaging and therapy. Transducers may be arranged in a variety of patterns within a panel, such as a rectilinear layout, a hexagonal pattern, an axially-oriented pattern, or a transversely-oriented pattern. In some embodiments, one or more of the panels 205 and 215 are not flat. For example, the panel 205 or 215 may comprise a curved configuration, wherein the entire panel is curved or a component of the panel, such as an array or tile (a group of transducers) of the panel is curved. The panels may comprise a shape that is convex inward. The panels 205 and 215 may comprise a flexible or semi-flexible shape. For example, at least part of the panel 205 or 215 may be configured such that it can be formed around an object (e.g., an arm).

Imaging and therapeutic transducers 210 and 220 may be arranged in a variety of relative positions. For example, in the embodiment of FIG. 2, the imaging and therapeutic panels 205 and 215 are alternately placed around the circumferential dimension of the cuff. In other embodiments, one or more imaging panels 215 are positioned adjacent to another imaging panel 215, and/or one or more therapeutic panels 205 are positioned adjacent to another therapeutic panel 205. In some embodiments, a panel 205 or 215 comprises at least one imaging transducer 220 and at least one therapeutic transducer 210. One or more panels may comprise no transducers.

A plurality of panels may share a characteristic, such as a shape characteristic or a material characteristic. For example, all of the panels of a device may be the same length in a dimension, such as the axial dimension. Two or more panels (such as all panels comprising a therapeutic transducer) may be the same length in the circumferential dimension.

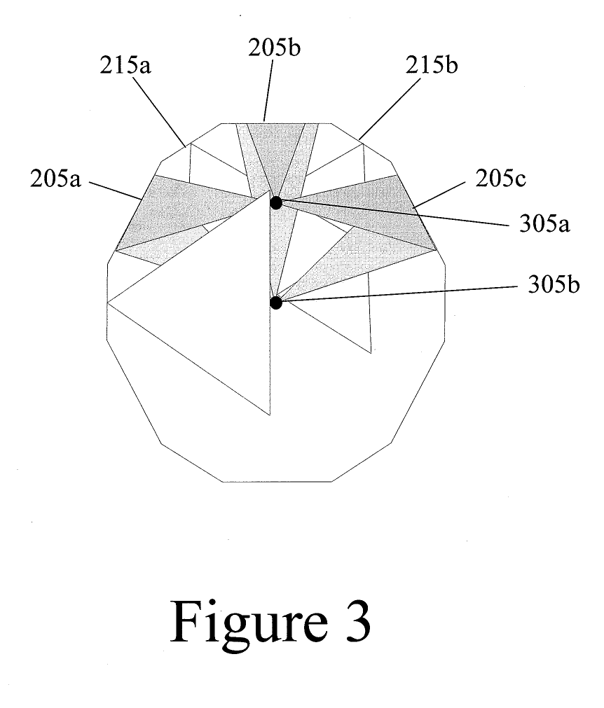

As described above, panels of a device may be positioned to form a cuff, as shown in FIG. 3. As shown in FIG. 3, the therapeutic panels 205 and the imaging panels 215 may be arranged and the therapeutic transducers driven such that the therapeutic transducers can selectively focus energy upon a target 305. As described in U.S. application Ser. No. 11/486,526, which is hereby incorporated by reference in its entirety, transducers and/or transducer arrays may selectively be activated, and selectively focused, based upon the determined location of a target. For example, FIG. 3 shows a shallow target 305a. The therapeutic transducers from panels 205a-c are selectively activated and focused, such that the ultrasound energy from the transducers from panels 205a-c are focused upon the target 305a. Ultrasound energy from a given therapeutic transducer array may be focused on the target 305a using a phased array or other focusing techniques known in the art. Meanwhile, the same transducers may be activated with a different driving scheme in order to focus the energy upon a deep target 305b. The shallow target 305a may be, for example, at least approximately 1, 2, 3, 4, 5, 6, 7, 8, 9, 10, 15, 20, or 30 cm from a therapeutic transducer (e.g., a transducer from panel 205b). The deep target 305b may be, for example, at least approximately 1, 2, 3, 4, 5, 6, 7, 8, 9, 10, 12, 13.5, 15, 20, or 30 from a therapeutic transducer (e.g., a transducer from panel 205b). The cuff may be symmetric about at least one axis.

Ultrasound systems, devices and methods disclosed herein may also include embodiments described in U.S. Application No. 60/973,658 or U.S. Application No. 60/699,253, which are hereby incorporated by reference in their entireties.

### ‘Point’-Like Position Sensors

The multiplicity of transducers and mechanical flexibility of the cuff can pose a significant treatment challenge since the relative spatial locations and acoustic direction orientations of each transducer, and here of each panel (either rigid or flexible), is advantageously known and communicated to the system control system, and/or to the operator, to enable appropriate targeting and to allow multiple therapeutic panels to be used to achieve, for example, efficient and robust hemostasis or tissue ablation. The relative spatial position and orientation definition of the entire multiplicity of panels (and respectively of the transducers within the panels), particularly relative to a target or structure, is here termed “device spatial registration.” Equivalent registration challenges exist for “blanket”, “patch” or other multiple-transducer configurations.

When the transducers are arranged in a cuff, an imaging panel 215 can localize a target, and this target position can be transformed to the local coordinates of multiple therapeutic panels 205 (to allow multiple beam treatment). Similarly, other imaging panels 215 may locate targets that are communicated to other imaging panel coordinates. One method to perform these transformations, and to achieve device spatial registration, is to use 3D volume images from each respective imaging transducer 220 to first define a coordinate system of each imaging transducer 220 (using imaging coordinate Ix-Ix transformation), and then manipulate the 3D images from each transducer 220 so as to “connect” all the images in a smooth and continuous and contiguous fashion, thereby connecting the respective coordinates systems of the transducers together in a known relationship. This technique, termed 3D image “stitching” can be complex to run, and the software to join or stitch the image data can be complex and time consuming to develop.

Accordingly, some embodiments provide a method that is conceptually simpler than using “stitching” for determining and communicating to the system device spatial registration information (e.g., the relative positions and orientations of the imaging transducers 220 with respect to each other, and with respect to the therapeutic transducer arrays 210, and with respect to a target 305 or structure). Embodiments can include ways to achieve correct transformation from one imaging coordinate system to another (if desired), or from each imaging (Ix) to the therapeutic (Tx) coordinate systems. These methods can apply to, for example, ultrasound cuff, blanket or partially enclosing distributed transducer designs, and are not restricted to the cuff concept in FIG. 3.

One such embodiment includes ‘point’-like acoustic transducer emitters and receivers (position sensors) located on the therapeutic and imaging panels 205 and 215 to determine the locations and orientations of the panels (and equivalently the transducers, here assumed co-planar with the flat panels). By “point”-like, it is meant that a relatively small single transducer or array of transducers are used such that emitted ultrasound energy is omni-directional (e.g., emitting spherical sound waves) and ultrasound energy can be detecting coming from angles other than normal to the transducer (e.g., having omni-directional sensitivity). It is not critical to have mechanically flat imaging transducer arrays or therapeutic transducer arrays. Only the relationship between the ‘point’-like emitters or receivers and the array on the common panel need be known. The ‘point’-like emitters or receivers may be separate sensors located on the respective rigid panels on a device or portions of imaging or therapeutic ultrasound arrays may be used as the ‘point’-like emitters or receivers. The sensors may comprise one or more small transducers (e.g., having a disk or square shape).

In some instances, an emitter is positioned on a first rigid component (e.g., a therapeutic panel) and a receiver on a second rigid component (e.g., an imaging panel) of a device, wherein the location of a target is known relative to the second panel. The location of the target relative to the first component may then be determined, for example, by a time-of-flight and/or triangulation process based on determining the distances between the sensors on the panels. For example, the distance between a ‘point’-like emitter and a ‘point’-like receiver may be determined based on the time it takes for a signal from the emitter to be detected by a receiver and on the speed of sound in the medium (i.e., time-of-flight). Repeating this procedure using a plurality of ‘point’-like emitters or receivers can provide complete relative location and position information of each rigid component using triangulation. The signal emitted by the emitter may be a short duration pulse or sequence of pulses (e.g., a coded sequence as discussed further below). In some instances, the location information may at least partly determine which transducers will transmit acoustic energy to treat the target and/or the beamforms (e.g., focal pattern) of the transmitted energy. Positioning information obtained from the position sensors may also be used with additional techniques to improve and/or verify the accuracy of the positioning information.

Emitters and/or receivers may be positioned on or over or mounted in a rigid device component, such as a transducer array, a panel, a tile, or a frame holding a panel or a tile. In some embodiments, the device component is flat, while in others, it is not. The emitters and/or receivers can either be individual elements within a device imaging (Ix) array or therapeutic (Tx) array component, or can be separate sensor transducers. If using individual elements of an Ix or Tx array for the sensor function, alternatively, the elements may be used within groups of elements (acting as discrete sensors), wherein the groups may operate in unison.

The sensors may be tilted in a device component, such as a panel, tile or subaperture. The tilt may reduce directivity losses that would otherwise occur without the tilt and/or enable preferential performance in specific directions (e.g., toward specific panels). For example, a tilted sensor may enable a receiver to receive a signal transmitted from an emitter on an adjacent panel. The sensors may be mechanically tilted. In some instances, the tilt of the sensors may be adjustable.

In some embodiments, one or more emitters are positioned on one, a plurality of, or all of the therapeutic panels 205. In some embodiments, one or more emitters are positioned on one, a plurality of, or all of the imaging panels 210. In some embodiments, one or more emitters are positioned on panels or other rigid components that do not comprise either therapeutic transducers or imaging transducers. In some embodiments, specific elements within imaging and/or therapeutic transducer arrays may be used as emitters and/or receivers.

Receivers may be positioned in a location that is known with respect to the emitter positions. The receiver positions may be measured and/or determined, statically and/or dynamically, relative to the emitter positions. In some embodiments, one or more receivers are positioned on one, a plurality of, or all of the therapeutic panels 205. In some embodiments, one or more receivers are positioned on one, a plurality of, or all of the imaging panels 210. In some embodiments, one or more receivers are positioned to panels that are neither therapeutic panels 205 nor imaging panels 210. In certain embodiments, each therapeutic panel 205 comprises a plurality of emitters, and each imaging panel 210 comprises a plurality of receivers. Therapeutic panels 205 may comprise both emitters and receivers. For example, a receiver of one therapeutic panel 205 may function to receive signals from an emitter of another therapeutic panel 205. Similarly, imaging panels 215 may comprise both emitters and receivers. In these instances, a single position sensor may function as both an emitter and a receiver or separate sensors may be used. Sensors that act as both emitters and receivers may provide advantages such as reducing the aperture area consumed by the sensors and/or simplifying system hardware. In some embodiments, imaging array elements, therapeutic array elements, imaging panels 215, and/or therapeutic panels 205 may comprise emitter and/or receiver sensors. It may be advantageous to use a plurality of emitters and/or receivers (e.g., on each panel), as the redundancy may reduce errors in the position measurements and calculations. Further, larger numbers of localization sensors (emitters, receivers, and/or transmit-receive elements) per panel may improve the accuracy and sensitivity of positioning information of a device. For example, time-of-flight information may be averaged across sensors and/or across transmissions to improve the sensitivity and accuracy of positioning information. However, it may be advantageous to separately calculate positioning information for small device components (such as tiles or small sub-apertures), in order to allow for increased flexibility of a device or panel. For example, time-of-flight triangulation between point-like sensors on small subunits of a device may allow for increased flexibility of a device or panel. As discussed above, transducers that are part of therapeutic or imaging arrays within the subunits may be used as the point-like sensors.

In some instances, emitters transmit a signal upon receiving a trigger. For example, emitters may be configured to transmit a signal upon the positioning of a device. In other instances, emitters transmit a signal randomly or regularly. The frequency of the transmission may be based at least partly on, for example, a number of emitters, a number of receivers, a desired accuracy of the positioning information, and/or an estimate of device movement. Larger frequencies may improve the accuracy of positioning information. Emitters may transmit signals at different times or at substantially the same time. In the latter case, the signals may be distinct among at least some of the emitters.

In some instances, receivers from a plurality of panels may receive a signal from an emitter. For example, receivers from a plurality of therapeutic panels 205 may receive a signal from an emitter on an imaging panel 215. This parallel processing technique may reduce and/or minimize the time required to localize the panels. Alternatively, one or more receivers from a single panel may receive the signal. In these instances, sensors from the receiving panel may transmit a signal to other panels (e.g., other therapeutic panels 205). Alternatively, a plurality of panels may be calibrated or known to be in a specific relationship, such that it is only necessary to determine position data relative to one of the panels.

An emitter may transmit a signal with a fixed or variable transmit voltage. The transmit voltage may be adjusted in accordance with the desired sensitivity of a system. For example, increased transmit voltage may result in increased overall sensitivity of a positioning component of a device. Transmit voltages may also be determined based upon the size of the device and/or the number or positions of the receivers.

In some embodiments, preamplifers are used to amplify a signal transmitted by an emitter. The preamplifier may be positioned adjacent to the emitter and/or the receiver. The preamplifier may increase the overall sensitivity of a positioning component of a device. In one example, a target may be localized within an image of an imaging transducer in an imaging panel (which may be considered mechanically flat, with imaging transducers co-planar with the panel and in known positions within the panel). The location of the target relative to therapeutic panels can be determined by placing small ‘point’-like emitters on each therapeutic panel whose acoustic emission can be detected by the imaging transducers, or discrete elements within the imaging panel. Alternatively, these emissions may be received by ‘point’-like receivers on the imaging panel, depending on the acoustic design of the imaging panel. In each case, the location of each therapeutic panels can be determined relative to the imaging panel using acoustic time-of-flight and acoustic triangulation principles. Since the target location is also determined relative to the imaging panel coordinates, a target location relative to each therapeutic panel can be calculated, and device spatial registration can be achieved.

In some embodiments, a ‘point’-like transmit and receive element is positioned on a device (e.g., a therapeutic panel of a device) disclosed herein. In these instances, a therapeutic panel may receive information regarding the position of other therapeutic panels, thereby providing information about what energy distribution would effectively interact with the energy from other panels to focus the energy upon a target.

The receivers and emitters disclosed herein may be used to improve the accuracy of previous methods and devices. The receivers and emitters can function to identify a position and orientation of a panel relative to another panel. Therefore, it may reduce the importance of aligning an imaging axis with a reference axis (such as an axis of the device). (A less precise alignment may be used in order to ensure that the entire region-of-interest or target is targeted by the device.) The reduced emphasis on alignment can reduce the errors that would occur from such alignment. Further, the devices may include improved flexibility, as the methods and devices disclosed herein may not require rigid relative positions of the panels of the device.

Embodiments disclosed herein may be used to determine one or more properties of the region being treated, which may enable determination of an effective ultrasound treatment characteristic. For example, a method may identify an effective longitudinal velocity and/or attenuation of a medium, such as a tissue. For example, in some embodiments, a signal emitted from one point-like sensor and detected by another point-like sensor may be used to determine the speed of sound between the two sensors or determine the signal attenuation through the medium between the sensors. Therapeutic and/or imaging performance parameters may then be tuned, for example, dynamically during treatment based on actual acoustic medium properties. When determining location or velocity information, the signal may comprise short duration pulses. However, longer duration signals may be used to determine attenuation characteristics. The frequency of the signal may be varied when determining attenuation.

In some instances, while it is desirable to transmit focused beams through a medium, the differences of the velocity of the beam within the medium can cause side lobes and degraded lateral resolution. Phase-correction algorithms can compensate for medium-induced errors in beamforming. The usage of ‘point’-like emitters/receivers can be used to identify characteristics of the beamform and alter the ultrasound signals transmitted by therapeutic transducers to compensate for any identified errors. Specifically, the emitters/receivers can be used to correct for phase aberration correction using maximum brightness or amplitude as well as time-reversal algorithms. The ‘point’-like acoustic emitters and receivers can also be used to measure medium sound speeds and attenuations, which can then be used to identify signals emitted from the therapeutic transducers that will effectively treat a target within a medium (e.g., a tissue).

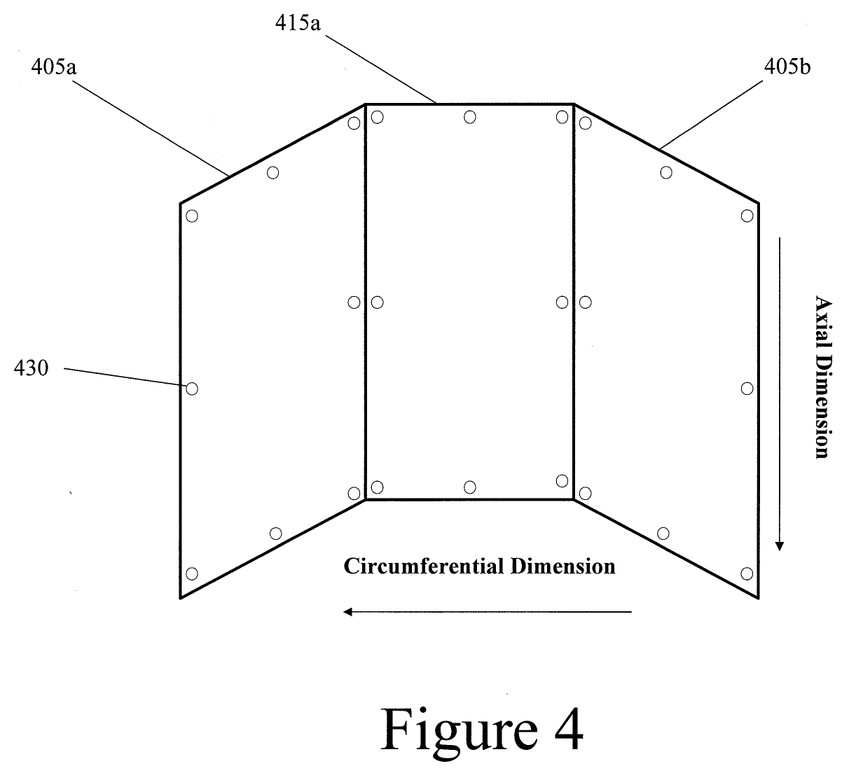

FIG. 4 shows one embodiment utilizing a cuff device. Three transducer panels are shown, two therapeutic panels 405a-b and one imaging panel 415. Each panel (imaging and therapeutic transducers not shown) has multiple acoustic localization sensors 430 (e.g., “point”-like transducers). If a target is localized with imaging panel 415a and therapy power is needed from the therapeutic panels 405a and 405b, the coordinates of the target relative to the two therapy panels can be determined. The target coordinate relative to imaging panel 415a may be given as (x1, y1, z1). Since the acoustic localization sensors for each panel are located (in this example) in a plane, determining the position of three points on a panel will provide localization. There are two scenarios that would allow the target relative to the therapeutic panels 405a and 405b to be determined.

### Scenario #1

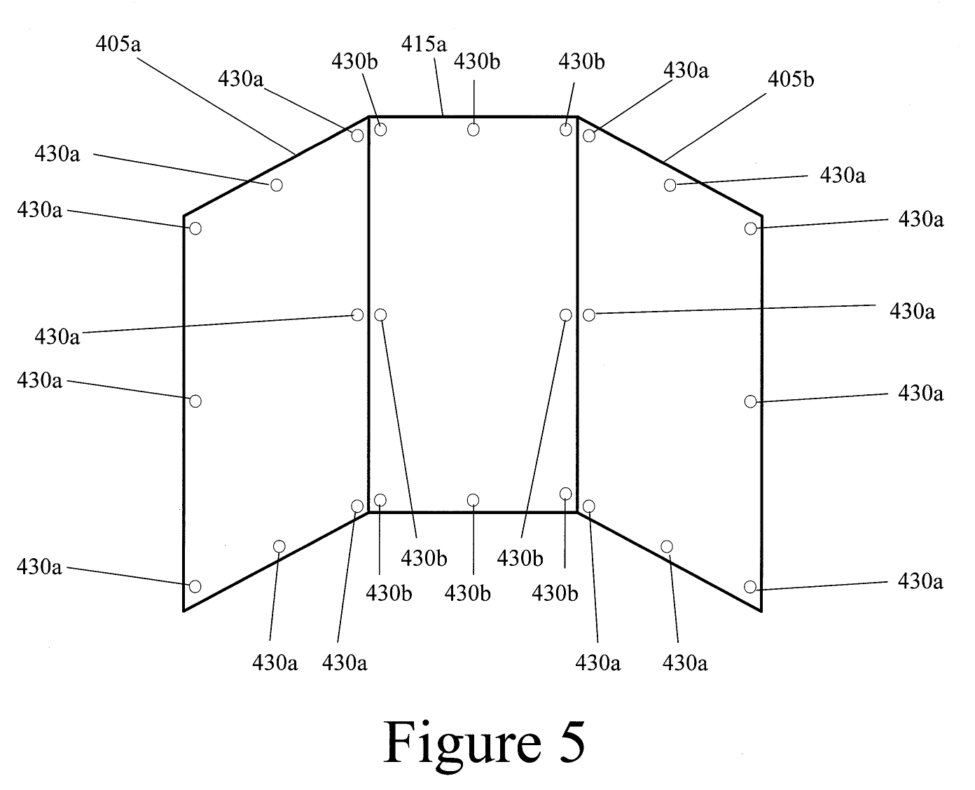

In one embodiment, depicted in FIG. 5, the imaging panel 415 has localization sensors 430 that are receivers only 430b and the therapeutic panels 405 have localization sensors 430 that are emitters only 430a. In this case, one of the acoustic localization emitter sensors 430a on the therapeutic panels 405 transmits. The signal is detected by localization receiver sensors 430b on the imaging panel 415. The coordinate of the emitter 430a is determined relative to the imaging panel 415 using any appropriate method, such as a time-of-flight and/or triangulation distance-measuring method. In some instances, only one signal is emitted from an emitter sensor 430a at a time. In other embodiments, a plurality of emitter sensors 430a may emit signals or pulses substantially simultaneously. These signals may be coded such that it is possible to distinguish, for example, as to which the panel of which the sensor was on. For example, the emitted signal may utilize a Barker or Golay code or be chirped. Such codes may include a pulse sequence having a unique signature in the time domain. A matched filter (e.g., a correlation filter) may be used at the receivers to analyze the pulses received and distinguish signals transmitted by different emitters. In one embodiment, the pulse waveform may be varied. A signal transmission process can continue until, for example, at least three points are determined and the plane of the therapeutic panel 405 is described in the Ix coordinate system. At this point, the emitters 430a of the therapeutic panel 405 may be transformed back into the Tx coordinate system along with the target position. This allows the proper beam profile and focus location from the therapeutic panel 405 to be used to treat the target.

### Scenario #2

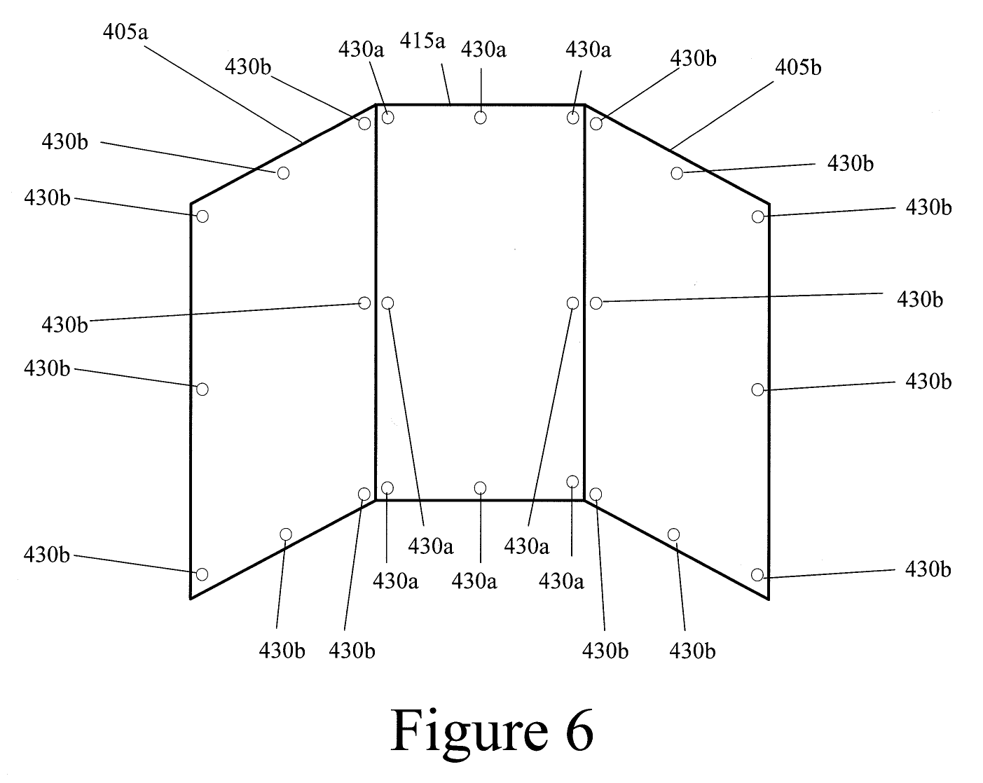

In another embodiment, depicted in FIG. 6, the imaging panel 415 has localization sensors 430 that are emitters only 430a and the therapeutic panels 405 have localization sensors 430 that are receivers only 430b. In this case, one of the acoustic localization emitter sensors 430a on the imaging panel 415 can transmit a signal. The signal can be detected by localization receiver sensors 430b on the therapeutic panels 405. The coordinate of the emitter 430a can be determined relative to the therapeutic panel 405 using, for example, a time-of-flight triangulation principle. At this point, the target position relative to the therapeutic panel 405 can be determined since the target is known relative to the imaging panel 415 just like the emitter 430a. If other (additional) emitters 430a from the imaging panel 415 are used, then possible error in the measurement may be reduced. Furthermore, the redundancy from multiple position sensors 430 may allow an estimate of the longitudinal velocity and attenuation of the medium. This scenario can be advantageous over Scenario #1 since fewer (e.g., only one transmit pulse) may be required.

In some embodiments, a device or system disclosed herein comprises a computer, a central control processor, and/or a central processing unit. Position information may be transmitted to the computer, central control processor, and/or a central processing unit. In some instances, the position information comprises the absolute location of an emitter or the location of an emitter relative to the location of a receiver. The position information may comprise an absolute or relative location of a imaging or therapeutic panel. The position information may comprise an absolute or relative location of a target. In one embodiment, the position information comprises the location of a target relative to a first panel and the location of a second panel relative to the first panel. The position information may comprise times. For example, the position information may indicate the time at which a particular signal was received. These times may be related to the distance between two points.

A device or system disclosed herein may comprise a computer configured to perform one or more steps of a process described herein. A device or system or a computer disclosed herein can include a microprocessor. The microprocessor can be any conventional general purpose single- or multi-chip microprocessor such as a Pentium® processor, Pentium II® processor, Pentium III® processor, Pentium IV® processor, Pentium® Pro processor, a 8051 processor, a MIPS® processor, a Power PC® processor, or an ALPHA® processor. In addition, the microprocessor can be any conventional special purpose microprocessor such as a digital signal processor. The microprocessor can have conventional address lines, conventional data lines, and one or more conventional control lines. The microprocessor can be configured to perform any process disclosed herein.

A device, system or computer disclosed herein can comprise a local area network (LAN). In one embodiment, the LAN conforms to the Transmission Control Protocol/Internet Protocol (TCP/IP) industry standard. In alternative embodiments, the LAN can conform to other network standards, including, but not limited to, the International Standards Organization's Open Systems Interconnection, IBM's SNA, Novell's Netware, and Banyon VINES.

A device, system or computer disclosed herein can include a memory. Memory refers to electronic circuitry that allows information, typically computer data, to be stored and retrieved. Memory can refer to external devices or systems, for example, disk drives or tape drives. Memory can also refer to fast semiconductor storage (chips), for example, Random Access Memory (RAM) or various forms of Read Only Memory (ROM), that are directly connected to the processor. Other types of memory include bubble memory and core memory.

A device, system or computer can include one or more input devices. For example, the input device can be a keyboard, rollerball, pen and stylus, mouse, or voice recognition system. The input device can also be a touch screen associated with an output device. A user can respond to prompts on the display by touching the screen. Textual or graphic information can be entered by the user through the input device.

A device, system or computer can comprise one or more output devices. The output device can include a display and/or screen. The output device can include a printer and/or a transmission component, by which the computer system can transmit data to another computer, a server or a network.

### Applications

In some instances, therapeutic ultrasound transducers described herein may be used to treat a target, which may comprise one or more of a blood vessel, an organ, a tumor, a fibroid, collagen, a wound, and trauma. A wound or trauma may have been caused by military combat, shrapnel wounds, a vehicular accident, civilian emergency, a penetrating device (e.g., a needle or catheter), or a medical procedure. The target may comprise a bleeding target, where it is desirable to terminate bleeding from the target location. When the target is a tumor or fibroid, the sultrasonic energy may be used to ablate or otherwise destroy the target.

While the above detailed description has shown, described, and pointed out novel features of the invention as applied to various embodiments, it will be understood that various omissions, substitutions, and changes in the form and details of the device or process illustrated can be made by those skilled in the art without departing from the spirit of the invention. The scope of the invention is indicated by the appended claims rather than by the foregoing description. All changes which come within the meaning and range of equivalency of the claims are to be embraced within their scope.

We claim:

1. An ultrasound device, comprising:
a first rigid component comprising an array of ultrasound transducers, the first rigid component comprising at least one ultrasound transducer configured to transmit an ultrasound pulse;
a second rigid component comprising an array of ultrasound transducers, the second rigid component comprising at least one ultrasound transducer configured to receive the transmitted pulse; and
a processor configured to determine the distance between the ultrasound transducer configured to transmit the ultrasound pulse and the ultrasound transducer configured to receive the transmitted pulse.

2. The device of claim 1, wherein the first rigid component comprises an array of ultrasound imaging transducers configured to detect a target and the second rigid component comprises an array of therapeutic transducers configured to transmit acoustic energy towards the target.

3. The device of claim 1, wherein the second rigid component comprises an array of ultrasound imaging transducers configured to detect a target and the first rigid component comprises an array of therapeutic transducers configured to transmit acoustic energy towards the target.

4. The device of claim 1, wherein the first rigid component comprises a plurality of ultrasound transducers configured to transmit ultrasound pulses and wherein the processor is configured to determine distances between the plurality of ultrasound transducers configured to transmit ultrasound pulses and the ultrasound transducer configured to receive based on measured time of flight of the pulses between the transmitter ultrasound transducers and the receiver transducers.

5. The device of claim 4, wherein the processor is configured to determine a position and orientation of the first rigid component relative to the second rigid component based on the determined distances.

6. The device of claim 5, wherein the processor is configured to determine said position and orientation using triangulation.

7. The device of claim 1, wherein the second rigid component comprises a plurality of ultrasound transducers configured to receive the transmitted pulse and wherein the processor is configured to determine distances between the plurality of ultrasound transducers configured to receive and the ultrasound transducer configured to transmit the ultrasound pulse.

8. The device of claim 7, wherein the processor is configured to determine a position and orientation of the first rigid component relative to the second rigid component based on the determined distances.

9. The device of claim 8, wherein the processor is configured to determine said position and orientation using triangulation.

10. The device of claim 1, wherein the first rigid component comprises an array of imaging transducers configured to detect a target and to transmit acoustic energy towards the target.

11. The device of claim 1, wherein the at least one ultrasound transducer configured to transmit an ultrasound pulse is part of the array of ultrasound transducers on the first rigid component.

12. The device of claim 1, wherein the at least one ultrasound transducer configured to receive the ultrasound pulse is part of the array of ultrasound transducers on the second rigid component.

13. The ultrasound device of claim 1, wherein at least one of the ultrasound transducer configured to transmit the pulse and the ultrasound transducer configured to receive the pulse is tilted with respect to a plane of the rigid component on which the tilted transducer is located.

14. The ultrasound device of claim 1, wherein the first rigid component is directly coupled to the second rigid component.

15. The ultrasound device of claim 1, wherein the processor is configured to determine a beamform to be transmitted by one of the arrays of ultrasound transducers based at least partly depending on the determined distance.

16. The ultrasound device of claim 1, wherein said ultrasound device comprises a cuff configured to be deployed circumferentially around a body limb, and wherein each rigid component comprises a panel in said cuff.

17. The ultrasound device of claim 16, further comprising a pressurizeable bladder positioned on a first side of the ultrasound arrays, wherein the cuff is configured such that when it is deployed circumferentially around the body limb, the bladder is positioned between the arrays and the body limb.

18. The ultrasound device of claim 1, wherein said ultrasound device comprises a device selected from an acoustic blanket, a conformal group of transducers, and a patch applicator, and wherein said array of ultrasound transducers are adapted to be deployed at least one of around and on a body part.

19. The ultrasound device of claim 18, wherein said body part comprises one or more of an abdomen, a head, a trunk.

20. The ultrasound device of claim 1, wherein each array of ultrasound transducers have substantially no curvature.

21. A method of transmitting focused ultrasonic energy, comprising:
determining a first location of a target relative to a first device component using ultrasound imaging;
emitting a signal from an ultrasound transducer located on a second device component;
detecting said signal at said first device component;
determining a second location of the target relative to the second device component based at least in part on the detected signal; and
applying high intensity focused ultrasonic energy from a therapeutic ultrasound transducer array located on the second device component to the target.

22. The method of claim 21, further comprising placing an ultrasound device comprising said first and second device components around or over a body part.

23. The method of claim 22, wherein said body part comprises a limb.

24. The method of claim 22, wherein said ultrasound device comprises an inflatable cuff.

25. The method of claim 21, wherein said ultrasonic energy is sufficient to cause hemostasis.

26. The method of claim 21, wherein said ultrasonic energy is sufficient to ablate tissue.

27. The method of claim 21, wherein the signal emitted from the ultrasound transducer located on the second device component is emitted in a plurality of directions.

28. The method of claim 21, further comprising:
emitting a plurality of signals from a plurality of ultrasound transducers located on said second device component; and
detecting said plurality of signals at said first device component, wherein said second location is determined based at least in part on the detecting of the plurality of signals.

29. The method of claim 21, wherein said target comprises a bleeding target.

30. The method of claim 21, wherein said target comprises a target selected from a tumor, a fibroid, a tissue target, and a collagen target.

31. The method of claim 21, wherein determining the second location comprises determining the time of flight of the emitted signal from the ultrasound transducer located on the second device component to the first device component.

32. A method of transmitting focused ultrasonic energy, comprising:
determining a first location of a target relative to a first device component using ultrasound imaging;
emitting a signal from an ultrasound transducer located on said first device component;
detecting said signal at a second device component;
determining a second location of the target relative to the second device component based at least in part on the detected signal; and
applying high intensity focused ultrasonic energy from a therapeutic transducer array located on the second device component to the target.

33. The method of claim 32, further comprising placing an ultrasound device comprising said first and second device components around or over a body part.

34. The method of claim 33, wherein said body part comprises a limb.

35. The method of claim 33, wherein said ultrasound device comprises an inflatable cuff.

36. The method of claim 32, wherein said ultrasonic energy is sufficient to affect hemostasis.

37. The method of claim 32, wherein said ultrasonic energy is sufficient to ablate tissue.

38. The method of claim 32, wherein the signal emitted from the second device component is emitted in a plurality of directions.

39. The method of claim 32, further comprising:
emitting a plurality of signals from a plurality of ultrasound transducers located on said first device component; and
detecting said plurality of signals at said second device component, wherein said second location is determined based at least in part on the detecting of the plurality of signals.

40. The method of claim 32, wherein said target comprises a bleeding target.

41. The method of claim 32, wherein said target comprises a target selected from a tumor, a fibroid, a tissue target, and a collagen target.

42. The method of claim 32, wherein determining the second location comprises determining the time of flight of the emitted signal from the ultrasound transducer located on the first device component to the second device component.

43. A method of transmitting focused ultrasonic energy, comprising:
positioning an ultrasound device comprising a therapeutic ultrasound array proximate to a human body;
emitting a signal from a first component of said ultrasound device into the body;
detecting said signal at a second component of said ultrasound device;
determining a speed of sound or signal attenuation in the body based at least in part on the detected signal; and
applying high intensity focused ultrasonic energy from said therapeutic transducer array into the body, wherein at least one characteristic of the energy is adjusted based on the determined speed of sound or signal attenuation.

44. A method of manufacturing an ultrasound device, comprising:
positioning an emitter configured to transmit an ultrasound signal on a first rigid device component; and
positioning a receiver configured to receive the transmitted ultrasound signal on a second rigid device component that is coupled directly or indirectly to the first rigid device component,
wherein the second rigid device component is movable with respect to the first rigid device component.

45. The method of claim 44, wherein said first device component comprises a therapeutic ultrasound transducer array and wherein said second device component comprises an imaging ultrasound transducer array.

46. The method of claim 44, wherein said first device component comprises an imaging ultrasound transducer array and wherein said second device component comprises a therapeutic ultrasound array.
